(19) United States
(12) Patent Application Publication
(10) Pub. No.: US 2024/0023872 A1
(43) Pub. Date: Jan. 25, 2024

(54) SYSTEMS AND METHODS OF OPTICAL NEURAL STIMULATION FOR INTRAOPERATIVE NERVE MONITORING

(71) Applicant: VANDERBILT UNIVERSITY, Nashville, TN (US)

(72) Inventors: Graham Throckmorton, Nashville, TN (US); Eric Duco Jansen, Nashville, TN (US); Anita Mahadevan-Jansen, Nashville, TN (US)

(21) Appl. No.: 18/223,785

(22) Filed: Jul. 19, 2023

Related U.S. Application Data
(60) Provisional application No. 63/390,355, filed on Jul. 19, 2022.

(51) Int. Cl. A61B 5/388; A61B 5/395

(52) U.S. Cl. CPC A61B 5/388; A61B 5/395

(57) ABSTRACT

Aspects of this invention relate to a system and method of neural stimulation for intraoperative nerve monitoring for a living subject. The system includes an optical source configured to generate light; a delivering means coupled to the optical source to deliver the generated light to a target nerve of the living subject for stimulating the target nerve; and a detector coupled to the target nerve to record evoked signals responsive to the stimulation for intraoperatively monitoring of the target nerve.

### CROSS-REFERENCE TO RELATED PATENT APPLICATION

This application claims priority to and the benefit of U.S. Provisional Patent Application Ser. No. 63/390,355, filed Jul. 19, 2022, which is incorporated herein by reference in its entirety.

### FIELD OF THE INVENTION

The invention relates generally to optical stimulation of bio objects, and more particularly, to systems and methods of optical neural stimulation for intraoperative nerve monitoring.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Iatrogenic nerve injuries (INI) have plagued surgical outcomes across all specialties. Between 450,000 and 600,000 INIs occur each year in the United States alone. In some cases like prostatectomies and mastectomies, the prevalence of INIs is reported to be as high as 85% and 60% respectively. The deleterious complications due to INI can range from numbness and loss of sensation to chronic pain and paralysis. Moreover, INIs are also a common source of medicolegal litigation with 60% of INI complications during thyroid surgery leading to malpractice lawsuits and 82% of cases of spinal accessory nerve injury resulting in patient compensation.

Diagnosis of INT is largely dependent on the surgeons' awareness of the injury and its symptoms that develop postoperatively. Consequently, intraoperative nerve monitoring (IONM) has been used since the late 1970s to alert surgeons to the onset of nerve damage and lower the incidence of INIs. IONM seeks to preserve peripheral nerve function through electrical stimulation (ES) of at risk nerves throughout surgery and examining any changes in the amplitude and latency of the evoked signals that are indicative of damage. By assessing nerve functionality throughout a surgical procedure, the risk of INT is greatly reduced and timely interventions can be made if damage occurs. Because IONM relies on ES, however, IONM suffers from several ES-based shortcomings.

First, ES requires contact with tissue to excite action potentials. Thus, changes in the degree of contact between the electrode and tissue can lead to misrepresentative evoked responses. Additionally, high frequency artifacts have long hindered proximal ES and electrophysiological recordings due to the superposition of the artifact onto the evoked signal. In the context of IONM, ES artifacts can often obscure the onset and alter magnitude of the evoked response making it impossible to accurately calculate the amplitude and latency needed to detect and prevent further damage. Lastly, ES is prone to current spread in which unconfined charge is distributed throughout the adjacent tissue. As a result, ES excites distant neural tissue beyond the intended target leading to potential misdiagnosis of nerve functionality and viability. Currently, surgeons are still searching for better stimulation techniques to improve the spatial resolution of

IONM.

Infrared neural stimulation (INS) is a label-free, optical method to excite neural tissue using pulsed infrared light (λ=1440-1550 nm and λ=1850-2120 nm). During INS, absorbed infrared light initiates action potentials via a thermally-mediated transient change in cell membrane capacitance which generates a depolarizing current. Due to its mechanism, INS is confined to small volumes dictated by the laser spot size and wavelength-dependent optical penetration, providing a high degree of innate spatial specificity. Consequently, the spatial precision of INS has been shown to selectively stimulate specific nerve fascicles, ocular dominance columns, and substructures of embryonic quail hearts among other targets. Additionally, owing to its unique mechanism, INS does not produce stimulation artifacts enabling simultaneous stimulation and recording in neighboring areas. Unlike its electrical counterpart, INS does not require contact with the target tissue. Human feasibility studies have shown that INS is a safe and effective means of clinical neurostimulation in the acute, intraoperative setting.

Therefore, it would be beneficial and desirable for surgeons to have new clinical tools of special relevance to the improvements of the IONM in the diagnosis of INT and/or in surgical procedures.

### SUMMARY OF THE INVENTION

In view of the foregoing, one of the objectives of this invention is to provide systems and methods of infrared neural stimulation as a potential clinical tool for intraoperative nerve monitoring. To directly compare INS to standard clinical ES, nerves were monitored using both modalities before and after partial or complete transection, crush, or stretch. In examining varying degrees of the three most prevalent INIs, INS outperforms ES exhibiting a higher sensitivity to less severe forms of damage due to its spatial selectivity. The efficacy of INS during IONM is also consistent across benchtop and clinical nerve monitoring systems. Improved sensitivity to less severe forms of injury could alert surgeons to the onset of damage earlier preventing further trauma and enabling timely interventions.

In one aspect of the invention, the system of neural stimulation for intraoperative nerve monitoring for a living subject comprising an optical source configured to generate light; a delivering means coupled to the optical source to deliver the generated light to a target nerve of the living subject for stimulating the target nerve; and a detector coupled to the target nerve to record evoked signals responsive to the stimulation for intraoperatively monitoring of the target nerve.

In one embodiment, the light source comprises a laser.

In one embodiment, the laser comprises a pulsed infrared laser.

In one embodiment, the light is pulsed infrared light having a wavelength in a range of about 1000-2500 nm, and a pulse duration in a range of about 1-10 ms.

In one embodiment, the pulsed infrared light has a pulse energy in a range of about 1-25 mJ with a radiant exposure in a range of about 0.1-3 J/cm².

In one embodiment, the delivering means is adapted for delivering the light directly to the target nerve at a distance away from the surface of the target nerve.

In one embodiment, the delivering means comprises a probe having one end coupled to the optical source for receiving the light therefrom and an opposite, working end for delivering the light to the target nerve, and wherein the working end is positioned at the distance away from the surface of the target nerve such that there is no object positioned between the working end of the probe and the target nerve.

In one embodiment, the distance is in a range of about 10-500 μm.

In one embodiment, the probe comprises one or more optical fibers, one or more wave guides, one or more channels, or a combination thereof.

In one embodiment, the delivering means further comprises a movable stage coupled to the probe for adjustably positioning the working end of the probe at the distance away from the target nerve.

In one embodiment, the movable stage comprises a micromanipulator.

In one embodiment, the delivering means comprises one or more optical mirrors, one or more optical lenses, one or more optical couplers, or a combination thereof, placed in an optical path for focusing and/or collimating the light onto the target nerve.

In one embodiment, the detector comprises at least one sensing electrode placed in a downstream muscle associated with the target nerve for recording the evoked signals.

In one embodiment, the detector is configured to record the evoked signals at a sampling rate in a range of about 5000-8000 Hz.

In one embodiment, the detector is further configured to process the evoked signals to obtain amplitudes and latencies of the evoked signals, wherein each amplitude is a difference between the maximum and minimum of each evoked signal, and wherein each latency is a duration from the peak of the stimulus to the peak of each evoked signal.

In one embodiment, the amplitudes and latencies are normalized to the mean of the corresponding baseline values.

In one embodiment, a ≥50% loss in a baseline amplitude and a ≥10% increase in a baseline latency serve as thresholds for neural damage detection.

In one embodiment, the evoked signals comprise compound muscle action potentials (CMAPs).

In another aspect of the invention, the method of neural stimulation for intraoperative nerve monitoring for a living subject comprising delivering light to a target nerve of the living subject at a distance away from the target nerve for stimulating the target nerve; recording evoked signals of the target nerve responsive to the stimulation; and processing the evoked signals for intraoperatively monitoring of the target nerve.

In one embodiment, the light is generated by an optical source including a pulsed infrared laser.

In one embodiment, the light is pulsed infrared light having a wavelength in a range of about 1000-2500 nm, and a pulse duration in a range of about 1-10 ms. In one embodiment, the pulsed infrared light has a pulse energy in a range of about 1-25 mJ with a radiant exposure in a range of about 0.1-3 J/cm².

In one embodiment, said delivering the light is performed by a probe having one end coupled to the optical source for receiving the light therefrom and an opposite, working end for delivering the light to the target nerve, and wherein the working end is positioned at a distance away from the surface of the target nerve such that there is no object positioned between the working end of the probe and the target nerve.

In one embodiment, the distance is in a range of about 10-500 μm.

In one embodiment, the probe comprises one or more optical fibers, one or more wave guides, one or more channels, or a combination thereof

In one embodiment, the working end of the probe is adjustably positioned at the distance away from the target nerve by a moveable stage.

In one embodiment, the movable stage comprises a micromanipulator.

In one embodiment, said delivering the light is performed by one or more optical mirrors, one or more optical lenses, one or more optical couplers, or a combination thereof, placed in an optical path for focusing and/or collimating the light onto the target nerve.

In one embodiment, said recording the evoked signals of the target nerve is performed by a detector having at least one sensing electrode placed in a downstream muscle associated with the target nerve for recording the evoked signals.

In one embodiment, the evoked signals of the target nerve is recorded at a sampling rate in a range of about 5000-8000 Hz.

In one embodiment, said processing the evoked signals comprises obtaining amplitudes and latencies of the evoked signals, wherein each amplitude is a difference between the maximum and minimum of each evoked signal, and wherein each latency is a duration from the peak of the stimulus to the peak of each evoked signal; and normalizing the amplitudes and latencies to the mean of the corresponding baseline values.

In one embodiment, a ≥50% loss in a baseline amplitude and a ≥10% increase in a baseline latency serve as thresholds for neural damage detection.

In one embodiment, the evoked signals comprise CMAPs. These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1 shows schematically a system of neural stimulation for intraoperative nerve monitoring for according to one embodiment of the invention.

FIG. 2 shows schematically a system of neural stimulation for intraoperative nerve monitoring for according to another embodiment of the invention.

FIG. 3 shows an experimental schematic for IONM for a rat sciatic nerve according to embodiments of the invention. CMAPs are recorded from the gastrocnemius and anterior tibialis muscles using a NIM® 2.0. A standard Prass monopolar stimulator probe is used to deliver ES from the NIM® 2.0. The Capella Nerve Stimulator serves as the infrared diode laser source for INS and is coupled to an INS probe for stimulation.

FIG. 4 shows schematically rat sciatic nerve preparations for the experiment shown in FIG. 3.

FIG. 5 shows schematically experimental procedures for comparing electrical and infrared neural stimulation for intraoperative nerve monitoring according to embodiments of the invention.

FIG. 6 shows that infrared neural stimulation is more sensitive to partial nerve transections according to embodiments of the invention. (Panel a) Normalized CMAP amplitudes resulting from ES in baseline, healthy, partial transection, and complete transection conditions. Black dashed line represents the amplitude damage threshold (50% decrease). (Panel b) Normalized CMAP amplitudes resulting from INS in baseline, healthy, partial transection, and complete transection conditions. (Panel c) The sensitivity and specificity for the amplitude-based IONM approach to transection injuries. (Panel d) Normalized CMAP latencies resulting from ES in baseline, healthy, partial transection, and complete transection conditions. Black dashed line represents the latency damage threshold (10% increase). If no CMAP was evoked latency was set to 1.2 for ease of visual interpretation. (Panel e) Normalized CMAP latencies resulting from INS in baseline, healthy, partial transection, and complete transection conditions. (Panel f) Sensitivity and specificity for the latency-based IONM approach to transection injuries. Specificity in all cases was calculated using the ‘Healthy’ category of responses. All data is normalized to the mean baseline values for each individual nerve. N=10 nerves for all data sets (5 rats).

FIG. 7 shows that infrared neural stimulation more readily detects crush injuries according to embodiments of the invention. (Panel a) Normalized CMAP amplitudes resulting from ES in baseline, healthy, partially crush, and complete crush conditions. Black dashed line represents the amplitude damage threshold (50% decrease). (Panel b) Normalized CMAP amplitudes resulting from INS in baseline, healthy, partially crush, and complete crush conditions. (Panel c) Sensitivity and specificity for the amplitude-based IONM approach to crush injuries. (Panel d) Normalized CMAP latencies resulting from ES in baseline, healthy, partial crush, and complete crush conditions. Black dashed line represents the latency damage threshold (10% increase). If no CMAP was evoked latency was set to 1.2 for ease of visual interpretation. (Panel e) Normalized CMAP latencies resulting from INS in baseline, healthy, partial crush, and complete crush conditions. (Panel f) Sensitivity and specificity for the latency-based IONM approach to crush injuries. Specificity in all cases was calculated using the ‘Healthy’ category of responses. All data is normalized to the mean baseline values for each individual nerve. n=10 nerves for all data sets (5 rats).

FIG. 8 shows that stretch injuries are revealed with similar efficacy using both electrical and infrared neural stimulation according to embodiments of the invention. (Panel a) Normalized CMAP amplitudes resulting from ES in baseline, healthy, partial stretch, and complete stretch conditions. Black dashed line represents the amplitude damage threshold (50% decrease). (Panel b) Normalized CMAP amplitudes resulting from INS in baseline, healthy, partial stretch, and complete stretch conditions. (Panel c) Sensitivity and specificity for the amplitude-based IONM approach to stretch injuries. (Panel d) Normalized CMAP latencies resulting from ES in baseline, healthy, partial stretch, and complete stretch conditions. Black dashed line represents the latency damage threshold (10% increase). If no CMAP was evoked latency was set to 1.2 for ease of visual interpretation. (Panel e) Normalized CMAP latencies resulting from INS in baseline, healthy, partial stretch, and complete stretch conditions. Note: Six normalized latency outliers in the partial stretch condition exceeded 4 (12-97) and were not plotted for better visualization. (Panel f) Sensitivity and specificity for the latency-based IONM approach to stretch injuries. Specificity in all cases was calculated using the ‘Healthy’ category of responses. All data is normalized to the mean baseline values for each individual nerve. n=10 nerves for all data sets (5 rats).

FIG. 9 shows that electrical stimulation and infrared neural stimulation produce consistent latencies and amplitudes in undamaged nerves over extended periods of time according to embodiments of the invention. (Panel a) Probability density function of normalized latencies produced in undamaged nerves resulting from ES (teal) and INS (red). Grayed region represents latency responses that exceed the damage threshold (10% increase in latency). (Panel b) Probability density function of normalized amplitudes evoked in undamaged nerves resulting from ES and INS. Grayed region represents amplitude values that fall below the damage threshold (50% decrease in amplitude). (Panel c) Statistical analysis of latency variance and false positive rate (Panel d) Statistical analysis of amplitude variance and false positive rate. (Panel e) Time course of normalized latency values over two hours produced by ES and INS. All values normalized to the mean at t=0min. Linear fitting performed on all data for ES (R₂=0.16) and INS (R²=0.01). (Panel f) Time course of normalized amplitude values over two hours produced by ES and INS. All values normalized to the mean at t=0 min. Linear fitting performed on all data for ES (R²=0.33) and INS (R²=0.004). n=30 nerves for all distribution graphs [panels a—d; 15 rats total]; n=3 nerves and rats for time course plots (panels e-f).

FIG. 10 shows that INS can be integrated in to existing clinical IONM systems while maintaining performance according to embodiments of the invention. (Panel a) Normalized CMAP amplitudes resulting from ES in baseline, healthy, partial transection, and complete transection conditions. Black dashed line represents the amplitude damage threshold (50% decrease). (Panel b) Normalized CMAP amplitudes resulting from INS in baseline, healthy, partial transection, and complete transection conditions. (Panel c) Sensitivity and specificity for the amplitude-based IONM approach to transection injuries. (Panel d) Normalized CMAP amplitudes resulting from ES in baseline, healthy, partial crush, and complete crush conditions. (Panel e) Normalized CMAP amplitudes resulting from INS in baseline, healthy, partial crush, and complete crush conditions. (Panel f) Sensitivity and specificity for the amplitude-based IONM approach to crush injuries. (Panel g) Normalized CMAP amplitudes resulting from ES in baseline, healthy, partial stretch, and complete stretch conditions. (Panel) Normalized CMAP amplitudes resulting from INS in baseline, healthy, partial stretch, and complete stretch conditions. (Panel i) Sensitivity and specificity for the amplitude-based IONM approach to stretch injuries. Specificity in all cases was calculated using the ‘Healthy’ category of responses. All data is normalized to the mean baseline values for each individual nerve. n=4 nerves for all data sets (2 rats).

FIG. 11 shows that electrical and infrared neural stimulation evoke consistent amplitudes over time as measured by clinical IONM system according to embodiments of the invention. (Panel a) Probability distribution of normalized amplitudes evoked in undamaged nerves resulting from ES (teal) and INS (red) using a clinical IONM system. Grayed region represents amplitude values that fall below the damage threshold (50% decrease in amplitude). (Panel b) Statistical analysis of amplitude variance and false positive rates. (Panel c) Time course of normalized amplitude values over two hours produced by ES and INS. All values normalized to the mean at t=0min. Linear fitting performed on all data for ES (R²=0.131) and INS (R²=0.026). n=12 nerves for all distribution graphs [panels a-b; 6 rats total]; n=3 nerves and rats for time course plot in (panel c).

FIG. 12 shows that nerve monitoring efficacy of infrared neural stimulation is dependent on its spatial selectivity according to embodiments of the invention. (Panel a) Illustration and representative CMAP traces from partially transected nerve. The blue trace and Spot 1 correspond to the stimulation of intact nerve fascicles resulting in a false negative (undamaged nerve). Black and gray traces correspond to upstream stimulation of damaged fascicles at the same radiant exposure as Spot 1 (gray) and at a higher radiant exposure (black). (Panel b) Illustration and representative CMAP traces from a partially crushed nerve. The blue trace and Spot 1 correspond to the stimulation of intact nerve fascicles resulting in a false negative. Black and gray traces correspond to upstream stimulation of damaged fascicles at the same radiant exposure as Spot 1 (gray) and a higher radiant exposure (black).

### DETAILED DESCRIPTION OF THE INVENTION

The present invention will now be described more fully hereinafter with reference to the accompanying drawings, in which exemplary embodiments of the present invention are shown. The present invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, the term “living subject” refers to a human being such as a patient, or a mammal animal such as a monkey.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

A wide range of surgical procedures require working in areas containing important nerve and neural structures. As a result, intraoperative nerve injury is a prevalent surgical risk and common source of medicolegal litigation. Currently, surgeons rely on their anatomical knowledge and naked-eye visualization of the surgical field to identify nerves. In particularly challenging cases like thyroidectomies and acoustic neuroma removal involving critical nerves surgeons employ intraoperative nerve monitoring (IONM) to assess nerve functionality during surgery and identify the onset of nerve damage. Even with IONM, the prevalence of nerve injury can be as high as 60% in some cases, and there are mixed results as to whether IONM reduces the risk of nerve damage.

Currently, electrical stimulation (ES) is used for IONM in commercial systems such as the NIM Nerve Monitoring System from Medtronic or Nerveana from Neurovision Medical Products. ES suffers from current spread in which the injected current disperses into the surrounding tissues and in some cases activates multiple nerves or fascicles beyond the target. ES also generates a stimulation artifact which can bleed into the recorded electrophysiological signals complicating or confounding amplitude and latency calculations (the metrics used to determine nerve damage).

In view of the foregoing, certain aspects of this inventions provide applications of infrared neural stimulation (INS) for IONM to accurately detect the onset of nerve damage. INS is a label-free optical means of exciting neural tissue using pulsed infrared light. Due to is biophysical mechanism. INS possesses an innate spatial specificity higher than that of traditional ES techniques. This innate spatial selectivity enables individual nerve fascicles to be stimulated generating isolated effector responses (e.g., muscle contractions). By stimulating and monitoring smaller populations of axons individually rather than the entirety of the nerve, the onset of nerve damage can be detected sooner and more evidently than with ES. Moreover, INS does not generate a stimulation artifact like ES. The absence of a stimulation artifact simplifies post-processing and increases confidence in amplitude and latency calculations.

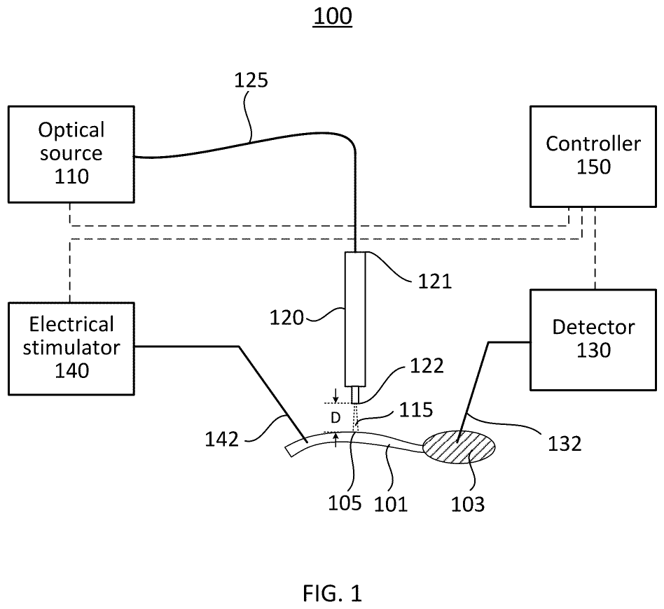

Referring to FIG. 1, a system 100 for optical stimulating neural tissue of a living subject is schematically shown according to one embodiment of the invention. The system 100 has a light source 110 capable of generating light, a probe 120 having a first end 121 optically coupled to the light source 110 through optical fibers or couplers 125 and a second end (i.e., working end) 122 for delivering the light 115 generated by the optical source 110 to a target nerve (neural tissue) 101. When the probe 120 delivers the light 115 to the target nerve 101 at a spot 105 through the working end 122, the working end 122 is positioned at a distance, D, away from the spot 105 of the surface of the target nerve 101 such that there is no object positioned between the working end 122 of the probe 120 and the target nerve 101. In some embodiments, the distance is in a range of about 10-500 μm.

In some embodiments, the light source 110 includes a solid state lasers (e.g. Ho:YAG and Er:YAG), CO₂ lasers, tunable OPO lasers, infrared LEDS, or infrared diode lasers that is capable of generating pulsed infrared light. The pulsed infrared light having a wavelength in a range of about 1000-2500 nm, a pulse duration in a range of about 1-10 ms, and a pulse energy in a range of about 1-25 mJ with a radiant exposure in a range of about 0.1-3 J/cm². In one exemplary embodiment shown in the EXAMPLE of the disclosure, the light source 110 is a 1450 nm diode laser (Capella, Lockheed Martin-Aculight, Bothell, Washington) coupled to a 400 μm core bare fiber (NA=0.22; Ocean Optics, Dunedin, Florida) for all INS experiments. The optical fiber was positioned orthogonal to the nerve surface using a micromanipulator (World Precision Instruments, Sarasota, Florida). In the exemplary embodiment, diode current is adjusted to deliver radiant exposure between 1.4 and 1.6 J/cm² at a pulse width of 500 μs. The stimulation radiant exposure is determined by incrementally increasing the diode current until a muscle twitch is achieved for every delivered pulse. Pulse trains lasting 10 s at a repetition rate of 2 Hz are employed for every nerve monitoring trial to minimize thermal superposition. The optical fiber is positioned to not be in contact with the tissue at distance of about 120 μm such that the average spot size at the tissue is 503.6±16 μm (1/e² diameter). It should be noted that other types of lasers can also be utilized to practice the invention.

In some embodiments, the probe 120 includes one or more optical fibers, one or more wave guides, one or more channels, or a combination thereof.

In some embodiments, the probe 120 is coupled to a movable stage that is movable three-dimensionally for adjustably positioning the working end 122 of the probe 120 at the distance away from the target nerve 101 and/or for selectively delivering the light 115 to one or more neural fibers or spots of the target nerve 101. In one embodiment, the movable stage comprises a micromanipulator.

As shown in FIG. 1, the system 100 also has a detector 130 coupled to the target nerve 101 to record evoked signals responsive to the stimulation for intraoperatively monitoring of the target nerve. The detector 130 comprises at least one sensing electrode 132 placed in a downstream muscle 103 associated with the target nerve 101 for recording the evoked signals, which are recorded at a sampling rate in a range of about 5000-8000 Hz. The evoked signals are processed by the detector 130 or a processor to obtain amplitudes and latencies of the evoked signals for IONM. In some embodiments, each amplitude is a difference between the maximum and minimum of each evoked signal, and wherein each latency is a duration from the peak of the stimulus to the peak of each evoked signal. The amplitudes and latencies are normalized to the mean of the corresponding baseline values.

In one embodiment, a ≥50% loss in a baseline amplitude and a ≥10% increase in a baseline latency serve as thresholds for neural damage detection.

In one exemplary embodiment shown in the EXAMPLE of the disclosure, the target nerve 101 is a sciatic nerve. The response of the sciatic nerve to the optical stimulation can be monitored using bipolar subdermal needle electrodes placed in either the tibialis anterior or soleus muscle for compound muscle action potential (CMAP) recording. In addition, The response of the sciatic nerve to the optical stimulation can be detected using subdermal needle electrodes placed under the sciatic nerve for compound nerve action potential (CNAP) recording.

In comparison, an electrical stimulator 140, which is also shown in FIG. 1 for comparison with the INS, is used to stimulate the target nerve 101, a stimulus is sent through stimulating electrodes 142 placed physically in contact with the target nerve 101 to elicit stimulation. The electrical stimulator 140, therefore, is not contact free and invasive. Sensing electrodes 132 are placed apart from the optical stimulation spot 105 and the electrical stimulating electrodes 142 to receive the action potentials generated by either the optical or electrical stimulation.

In some embodiment, the system 100 may optionally include a controller 150, such as a computer or the like, operably coupled with the light source 110, the probe 120, the detector 130, and/or the electrical stimulator 140 to synchronize the operations of them and/or to conduct data analysis.

Furthermore, the system 100 can be integrated into existing commercial IONM systems as a substitute for ES.

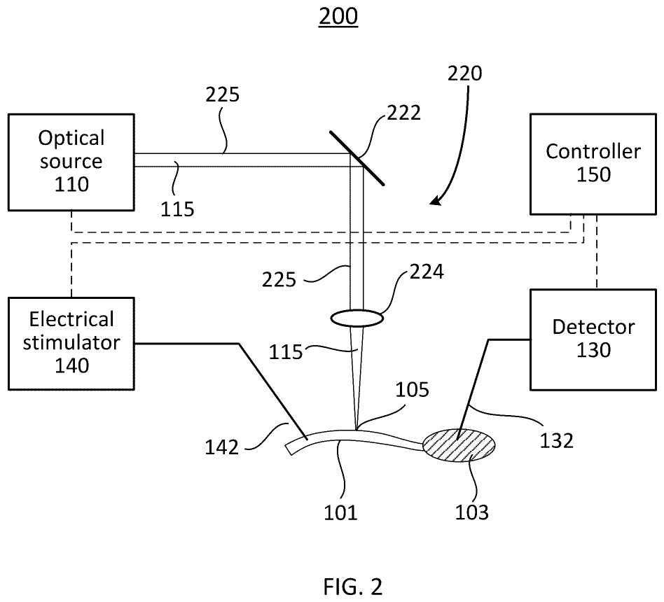

Referring now to FIG. 2, the system 200 for optical stimulating neural tissue of a living subject is shown according to another embodiment of the invention. The system 200 is substantially similar to the system 100 shown in FIG. 1, except that the optical delivering means 220 delivers the light 115 to the target nerve 101 in a contact-free manner.

In some embodiments, the delivering means 220 includes one or more optical reflectors/mirrors, one or more optical lenses, one or more optical couplers, or a combination thereof, placed in an optical path 225 for focusing and/or collimating the light 115 onto the target nerve 101.

In the exemplary embodiment shown in FIG. 2, the delivering means 220 has a first optical means 222 for receiving the light 115 from the light source 110 along optical path 225 and then directing the light 115 along optical path 225 to a second optical means 224 for focusing the light 115 directed by the first optical means 222 to the target nerve 101. The light 115 arrives at the target nerve 101 at a selected spot 105 to cause optical stimulation. In the embodiment shown in FIG. 2, the first optical means is an optical reflector/mirror 222. The second optical means is an optical lens 224.

It should be note that other configurations of the delivering means or probes to deliver the light to the target nerve may also be utilized to practice the invention.

In another aspect of the invention, the method of neural stimulation for intraoperative nerve monitoring for a living subject comprising delivering light to a target nerve of the living subject at a distance away from the target nerve for stimulating the target nerve; recording evoked signals of the target nerve responsive to the stimulation; and processing the evoked signals for intraoperatively monitoring of the target nerve.

In some embodiments, the light is generated by an optical source including a pulsed infrared laser.

In some embodiments, the light is pulsed infrared light having a wavelength in a range of about 1000-2500 nm, and a pulse duration in a range of about 1-10 ms.

In some embodiments, the pulsed infrared light has a pulse energy in a range of about 1-mJ with a radiant exposure in a range of about 0.1-3 J/cm².

In some embodiments, said delivering the light is performed by a probe having one end coupled to the optical source for receiving the light therefrom and an opposite, working end for delivering the light to the target nerve, and wherein the working end is positioned at a distance away from the surface of the target nerve such that there is no object positioned between the working end of the probe and the target nerve.

In some embodiments, the distance is in a range of about 10-500 μm.

In some embodiments, the probe comprises one or more optical fibers, one or more wave guides, one or more channels, or a combination thereof.

In some embodiments, the working end of the probe is adjustably positioned at the distance away from the target nerve by a moveable stage.

In some embodiments, the movable stage comprises a micromanipulator.

In some embodiments, said delivering the light is performed by one or more optical mirrors, one or more optical lenses, one or more optical couplers, or a combination thereof, placed in an optical path for focusing and/or collimating the light onto the target nerve.

In some embodiments, said recording the evoked signals of the target nerve is performed by a detector having at least one sensing electrode placed in a downstream muscle associated with the target nerve for recording the evoked signals.

In some embodiments, the evoked signals of the target nerve is recorded at a sampling rate in a range of about 5000-8000 Hz.

In some embodiments, said processing the evoked signals comprises obtaining amplitudes and latencies of the evoked signals, wherein each amplitude is a difference between the maximum and minimum of each evoked signal, and wherein each latency is a duration from the peak of the stimulus to the peak of each evoked signal; and normalizing the amplitudes and latencies to the mean of the corresponding baseline values.

In some embodiments, a ≥50% loss in a baseline amplitude and a ≥10% increase in a baseline latency serve as thresholds for neural damage detection.

In some embodiments, the evoked signals comprise CMAPs.

In sum, the INS can be used as a clinical tool for IONM. The INS is a label-free optical means of exciting neural tissue using pulsed infrared light. Due to is biophysical mechanism, INS has a higher degree of spatial precision than that of traditional electrical stimulation (ES) techniques. This innate spatial selectivity enables individual nerve fascicles to be stimulated generating isolated effector responses (e.g., muscle contractions). By stimulating specific fascicles rather than the entirety of the nerve, the onset of nerve damage can be detected earlier. Moreover, INS does not produce a stimulation artifact further simplifying latency and amplitude calculations and increasing confidence in measured signals.

These and other aspects of the invention are further described below. Without intent to limit the scope of the invention, exemplary instruments, apparatus, methods and their related results according to the embodiments of the invention are given below. Note that titles or subtitles may be used in the examples for convenience of a reader, which in no way should limit the scope of the invention. Moreover, certain theories are proposed and disclosed herein;

however, in no way they, whether they are right or wrong, should limit the scope of the invention so long as the invention is practiced according to the invention without regard for any particular theory or scheme of action.

### EXAMPLE

### Infrared Neural Stimulation Markedly Enhances Nerve Functionality Assessment During Nerve Monitoring

In surgical procedures where the risk of accidental nerve damage is prevalent, surgeons commonly use electrical stimulation (ES) during intraoperative nerve monitoring (IONM) to assess a nerve's functional integrity. ES, however, is subject to off-target stimulation and stimulation artifacts disguising the true functionality of the specific target and complicating interpretation. Lacking a stimulation artifact and having a higher degree of spatial specificity, infrared neural stimulation (INS) has the potential to improve upon clinical ES for IONM.

In this exemplary example, a direct comparison between clinical ES and INS for IONM performance in an in vivo rat model is presented. The sensitivity of INS surpasses that of ES in detecting partial forms of damage while maintaining a comparable specificity and sensitivity to more complete forms. Without loss in performance, INS is readily compatible with existing clinical nerve monitoring systems. These findings underscore the clinical potential of INS to improve IONM and surgical outcomes.

Specifically, we demonstrate the application of INS as a potential clinical tool for IONM in an in vivo sciatic nerve model. To directly compare INS to standard clinical ES, nerves were monitored using both modalities before and after partial or complete transection, crush, or stretch. In examining varying degrees of the three most prevalent INIs, INS outperforms ES exhibiting a higher sensitivity to less severe forms of damage due to its spatial selectivity. The efficacy of INS during IONM is also consistent across benchtop and clinical nerve monitoring systems. Improved sensitivity to less severe forms of injury could alert surgeons to the onset of damage earlier preventing further trauma and enabling timely interventions.

### Materials And Methods

Animal preparation: All experiments were conducted at the Vanderbilt Biophotonics Center in adherence to protocols approved by the Vanderbilt Institution of Animal Care and Use Committee (IACUC) and are reported in accordance with ARRIVE guidelines where applicable.

All methods were performed according to the relevant ethical guidelines and regulations as approved by the Vanderbilt IACUC. The surgical preparations used here have been described elsewhere in detail. Briefly, adult male and female Sprague—Dawley rats (250-300 g) were anesthetized and maintained under sedation using isoflurane (n=30 total). To expose the sciatic nerve and its trifurcations, a about 3 cm incision was made on the lateral side of the leg extending from the gluteus to popliteal region using a split-muscle technique. Room temperature sterile saline was routinely applied to the nerve throughout experiments to maintain tissue hydration and prevent desiccation.

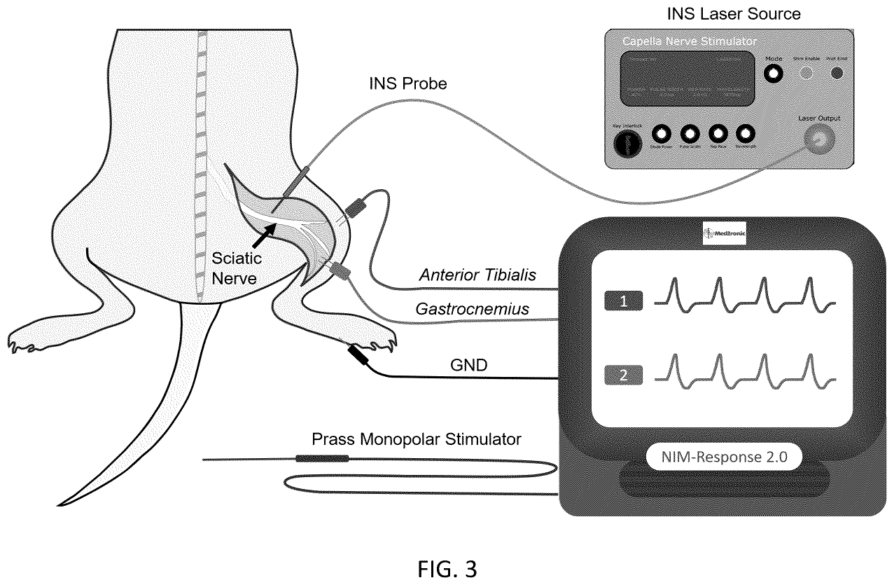

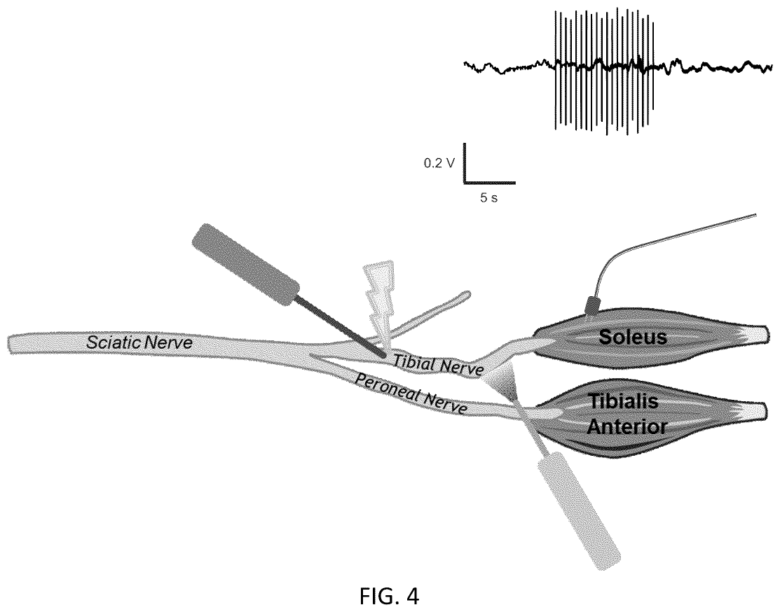

Benchtop electrophysiology: Experiments utilizing research grade equipment were performed with a modular data acquisition system (MP100, Biopac Systems Inc., Santa Barbara, California) to simultaneously record evoked CMAPs and INS or ES triggering, as shown in FIG. 3. This enabled accurate calculation of latencies values resulting from both INS and ES. For experiments on the common peroneal and tibial nerve, paired, bipolar subdermal needle electrodes (Medtronic Xomed, Jacksonville, Florida) were placed in either the tibialis anterior or soleus muscle respectively to record evoked activity, as shown in FIGS. 3-4. Both a subdermal grounding electrode and stimulus return were also inserted into the foot on the ipsilateral leg with the ground more proximal to the location of the applied stimulus. Evoked signals were sampled at a rate of 6500 Hz, amplified 1000×, and bandpass filtered from 0.05 to 5000 Hz with a differential amplifier (DA 100C, Biopac Systems Inc., Santa Barbara, California).

Clinical electrophysiology: A NIM-Response 2.0 (Medtronic, Minneapolis, Minnesota) was used in experiments demonstrating INS compatibility with existing clinical IONM systems, as shown in FIG. 3. Just as with the benchtop system, bipolar subdermal needle electrodes were placed in either the tibialis anterior or soleus muscle. Additional subdermal electrodes were used for grounding and stimulus return and placed in the ipsilateral foot. NIM-Response 2.0 bandpass filtering was internally fixed by the manufacturer at 100-2000 Hz. The electrical stimulation artifact delay on the NIM-Response 2.0, used as means to work around the stimulation artifact, was set to 3.1 ms.

Infrared neural stimulation: A 1450 nm diode laser (Capella, Lockheed Martin-Aculight, Bothell, Washington) coupled to a 400 μm core bare fiber (NA=0.22; Ocean Optics, Dunedin, Florida) was used for all INS experiments. The optical fiber was positioned orthogonal to the nerve surface using a micromanipulator (World Precision Instruments, Sarasota, Florida). In accordance with previous optimization studies, diode current was adjusted to deliver radiant exposure between 1.4 and 1.6 J/cm² at a pulse width of 500 μs. The stimulation radiant exposure was determined by incrementally increasing the diode current until a muscle twitch was achieved for every delivered pulse. Pulse trains lasting 10 s at a repetition rate of 2 Hz were employed for every nerve monitoring trial to minimize thermal superposition. The optical fiber was positioned to not be in contact with the tissue at distance of about 120 μm such that the average spot size at the tissue was 503.6±16 μm (1/e² diameter) as measured by an infrared beam profiler (BP209-IR2, Thorlabs, Newton, New Jersey) and validated using the knife-edge technique.

Electrical stimulation: A standard Prass monopolar stimulator probe (Medtronic Xomed, Jacksonville, Florida) was used for all experiments. When using the benchtop system, monophasic, square pulses with currents <1 mA were used to evoke CMAPs. To match INS stimulation parameters, ES nerve monitoring trials with the benchtop system were performed with a pulse width of 500 μs at frequency of 2 Hz. Trials using the NIM-Response 2.0 system the pulse width was set to 100 μs and the frequency to 4 Hz while maintain a stimulus <1 mA. The ES threshold was determined by incrementally increasing the applied current until a muscle twitch was induced for each delivered stimulus.

Nerve injury: Three types of INI were investigated in this study: transection, crush, and stretch. Each was examined at two degrees of severity: a partial and complete form. Transection injuries were inflicted by cutting through approximately half of and the entirety of the nerve's diameter for the partial and complete forms of damage respectively. Partial transection injuries were inflicted using a 3D printed nerve cutting guide fitted with a semicircular nerve notch to hold the nerve in place. One guide had nerve notch with a diameter equal to the average diameter of the rat common peroneal nerve (0.4 mm) and other the average diameter of the tibial nerve (0.63 mm). Both guides were fitted with a blade tract that directed the razor blade to make a transverse cut through half the diameter of the respective nerve.

Crush injuries were made perpendicular to the axis of the nerve using Kelly hemostats with a closing force of 1.12 N. Complete crush injuries were made by crushing the entire diameter of the nerve. For partial crush injuries, only half of the diameter of the nerve was crushed. For both partial and complete crush injuries, the hemostats were left clamped to the nerve for 10 s duration to allow stable compression.

For stretch injuries, the nerve was put into tension using hook electrodes mounted to a micromanipulator. To measure strain, two dye markers were placed on the surface of the nerve before insult, and the distance between the two was measured with calipers (0.1-1 cm). After the nerve was stretched using the micromanipulator, the distance between the dye markers was measured again and used to calculate the induced strain. Partial stretch injuries resulted in an average induced strain of 8.62±1.6 and 13.4±3.6% for complete stretch injuries. There was no difference in the pulling strength between partial and complete conditions. The induced strains were chosen based on the work of Rickett et al. With respect to partial stretch injuries, Rickett et al. observed the minimum threshold for functional deficit after a nerve was stretched between 5 and 10%. This study was also supported by Driscoll et al. and Li and Shi who also reported functional deficits at 8.8 and 8.3% respectively. Similarly, for the complete stretch condition strains greater than 10% were chosen as Rickett et al. observed that the mechanical tolerance of the nerve was exceeded above 10%.

Data analysis: As the current clinical standard, a ≥50% loss in baseline amplitude and a ≥10% increase in the baseline latency served as the thresholds for neural damage detection. Amplitude was defined as the difference between the maximum and minimum of the evoked response to eliminate the need for any baseline corrections in the recordings. Latency was defined as the duration from the peak of the stimulus to the peak of the evoked response. For all trials, “Healthy”, “Partial”, and “Complete” amplitudes and latencies were normalized to the mean of the corresponding baseline values from individual experiments. In benchtop experiments, only trials that resulted in a clear separation of the ES artifact and evoked CMAP were considered in data analysis in order to accurately quantify amplitude and latencies.

Statistical analysis: All datasets were tested for normality using a Kolmogrov-Smirnov test. As most distributions were not normal, equivalence of variance was evaluated using an Ansari-Bradley test.

All sensitivity calculations were made utilizing CMAP amplitudes and latencies obtained after nerve injury using the standard formula:

$$Sensitivity = \frac{True Positives}{True Positives + False Negatives}$$

where true positives correspond to values correctly classified as damaged responses (i.e., an amplitude <50% or a latency >110% of their respective baseline values) and false negatives correspond to values incorrectly classified as healthy responses (i.e., an amplitude >50% or a latency <110% of their respective baseline values). Since these values were obtained after the nerve was injured, the true positives represent the accurate classification of the nerve as damaged while the false negatives inaccurately indicate that the nerve is undamaged/healthy. Similarly, specificity was calculated using CMAP amplitude and latencies elicited after baseline acquisition and prior to nerve injury (i.e., while the nerve remained healthy and undamaged). Specificity was calculated using the standard formula:

$$Specificity = \frac{True Negatives}{True Negatives + False Positives}$$

where true negatives correspond to values correctly classified as healthy responses (i.e., an amplitude >50% or a latency <110% of their respective baseline values) and false positives correspond to values incorrectly classified as damaged responses (i.e., an amplitude <50% or a latency >110% of their respective baseline values). Since these values were obtained before the nerve was injured, the true negatives represent the accurate classification of the nerve as undamaged and healthy while the false positives inaccurately indicate that the nerve is damaged. This calculation was identical for both partial and complete forms of injury, amplitude and latency metrics, benchtop and clinical IONM systems, and across all three injury types.

All false positive rates were calculated using the amplitude and latencies obtained from undamaged nerves and the standard formula:

$$False Positive Rate = \frac{False Positives}{False Positives + True Negatives}$$

Since the false positive rate is only calculated in healthy, undamaged nerves, false positives correspond to amplitudes and latencies that incorrectly indicated the nerve was damaged (i.e., amplitudes <50% or latencies >110% their respective baseline values). Accordingly, true negatives correspond to amplitudes and latencies that correctly indicated that the nerve was undamaged (i.e., amplitudes >50% or latencies <110% their respective baseline values).

### Results

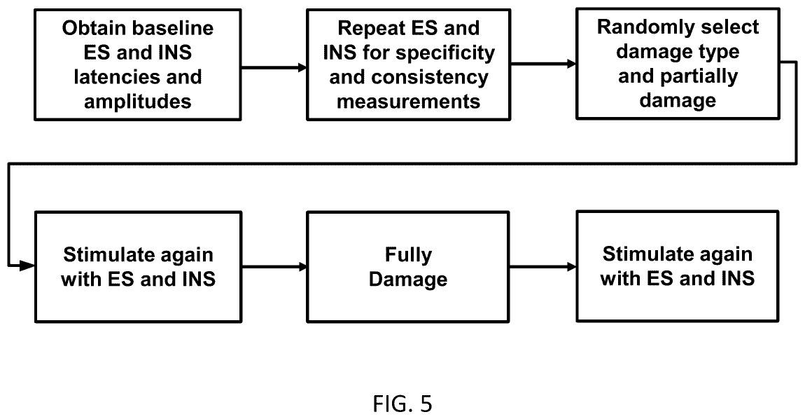

To directly compare ES and INS, an in vivo rat sciatic nerve preparation was utilized to examine each technique's ability to detect different forms and degrees of nerve injury (Table 1). The rat sciatic nerve along with its trifurcations share similar diameters (0.25-0.90 mm) to that of human nerves such as the recurrent laryngeal (0.71-2.0 mm) and facial nerve (1.1-2.6 mm) which are commonly monitored intraoperatively. As shown in FIG. 5, baseline compound muscle action potential (CMAP) amplitude and latency values were acquired for both ES and INS at the beginning of each trial from either the common peroneal or tibial nerve branch. After about 10 min, another set of amplitude and latency measurements were obtained with both techniques to ensure reproducibility of baseline, healthy values. To assess each modality's sensitivity to injuries of varying severity, the interrogated nerve was then partially damaged with a transection, crush, or stretch injury and then stimulated again. These three types of injury are the most common types of INIs and are representative of all three Seddon classifications. Lastly, the nerve was completely damaged and stimulated again to acquire amplitude and latency values.

As the current clinical standard, a ≥50% loss in baseline amplitude and a ≥10% increase in the baseline latency serve as the thresholds for neural damage detection. Once completed, the entire protocol was repeated for the remaining sciatic nerve branch.

TABLE 1
Examined nerve injuries, methods, extent, and Seddon classification.

| | | Partial injury | | Complete injury | |
|---|---|---|---|---|---|
| | | | Seddon | | Seddon |
| Injury type | Method | Extent | classification | Extent | classification |
| Transection | Razor blade | Half the | Neurotmesis | Cut through | Neurotmesis |
| | via nerve | diameter of | | entirety of the | |
| | cutting guide | the nerve | | nerve | |
| | | severed | | | |
| Crush | Calibrated | Half of the | Axonotmesis | Crushed entire | Axonotmesis |
| | hemostats | nerve diameter | | diameter of | |
| | | crushed | | the nerve | |
| Stretch | Hook | Nerve | Neuropraxia | Stretched | Neuropraxia |
| | electrodes | stretched to an | | nerve to an | |
| | mounted to | average strain | | average strain | |
| | micro- | of: ε = 8.6 ± | | of: ε = 13.4 ± | |
| | manipulator | 1.6% | | 3.6% | |

### Partial Nerve Transections Are More Reliably Detected By Infrared Neural Stimulation

To determine whether INS offers any benefit in identifying transections, nerves were partially and completely severed while using INS and ES for IONM. A 3D printed nerve cutting guide was used to cut through approximately half the nerve's diameter (41-59%) perpendicular to its long axis with a razor blade. The nerve was entirely severed for the complete form of transection injury. After baseline values were collected, the undamaged nerves were restimulated to ensure values were consistent (designated as the ‘Healthy’ condition in all figures) and to obtain a specificity.

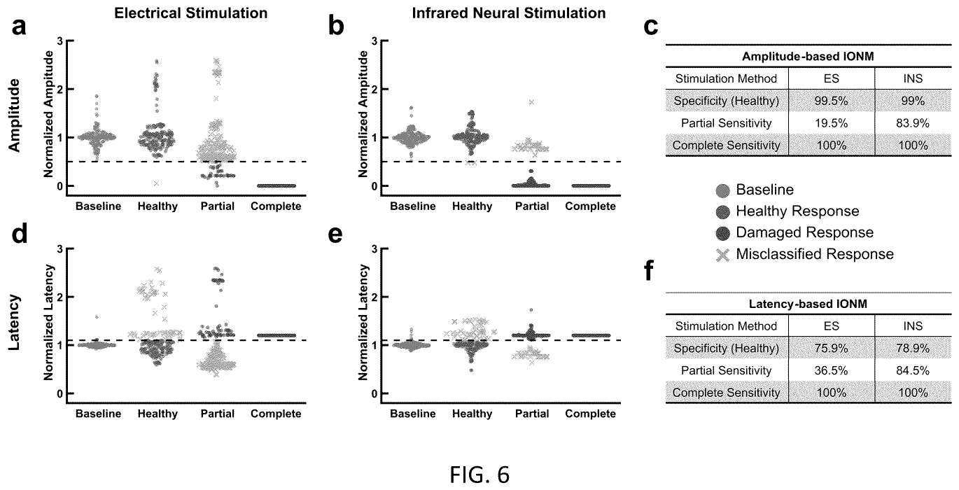

The specificity of INS and ES are nearly equivalent for both amplitude and latency-based approaches (panels c and f of FIG. 6). However, ES exhibits a broader amplitude distribution in the healthy condition (panel a of FIG. 6) than INS whose healthy distribution nearly replicates that of the baseline values (panel b of FIG. 6). Moreover, ES largely fails to indicate the presence of a partial transection (Sensitivity=19.5%) while INS detects the injury the majority of the time (Sensitivity=83.9%; panels a-b of FIG. 6). This is also consistent for latency-based IONM (panels d-e of FIG. 6). Trials in which INS misclassified partial transection as healthy are a result of its spatial selectivity.

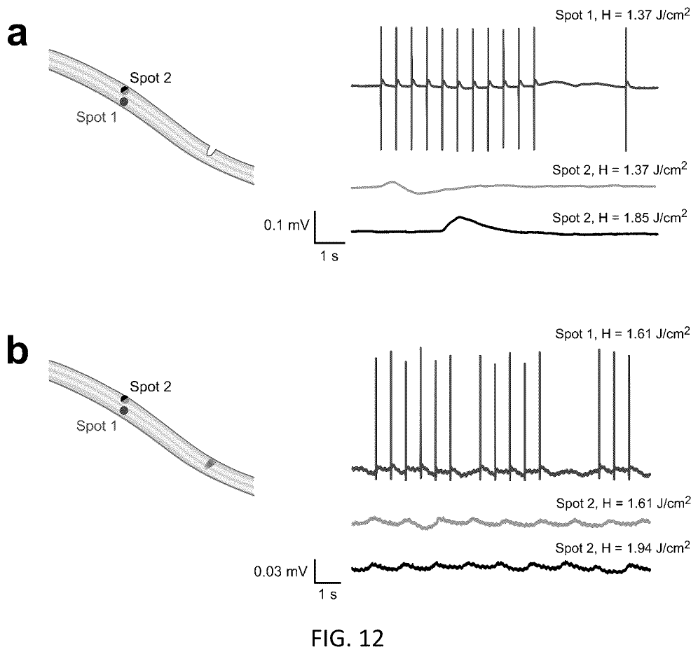

All nerves were damaged distally to the point of trifurcation and stimulated proximally. Trials in which INS did not detect nerve partial transections activated fascicles whose distal segments remained in continuity rather than activating fascicles whose distal segments had been transected (panel a of FIG. 12). This was confirmed by translating the fiber across the nerve and observing the corresponding loss of CMAPs even at higher radiant exposures (panel a of FIG. 12). Both methods exhibit equal sensitivities to complete transections as no action potential propagation is possible.

Infrared neural stimulation is more sensitive to crush injuries Crush injuries were inflicted by transversely applying calibrated hemostats to the nerve.

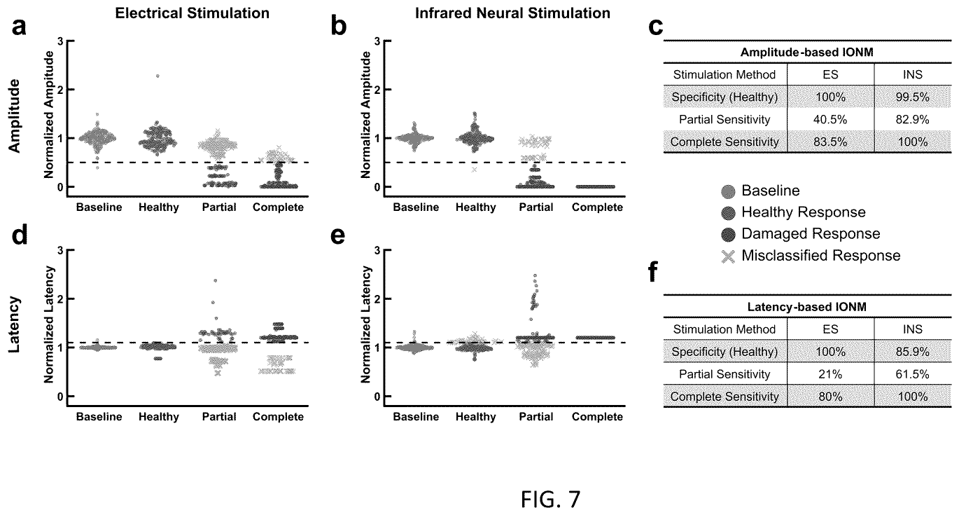

For partial crush injuries, only half the diameter of the nerve was crushed. The entire diameter of the nerve was crushed for complete crush injuries. In the amplitude-based IONM, the healthy distribution for INS again closely recapitulates that of the baseline whereas ES healthy distribution broadens (panels a-b of FIG. 7). This trend, however, is not as apparent for latency-based IONM in which INS exhibits a broader distribution (panels d-e of FIG. 7). (Variance of baseline and healthy condition values is thoroughly examined in a subsequent section). Unlike with transection injuries, INS is more sensitive to partial and complete crush injuries. For both amplitude- and latency-based IONM, INS exhibits over a two-fold increase in sensitivity for the partial crush condition (panels c and f of FIG. 7). In full crush experiments, INS successfully detected all damaged nerves (panels b and e of FIG. 7) whereas ES failed to recognize these injuries in about 20% of the trials panels a and d of (FIG. 7). Similar to the partial transections, INS fails to recognize partial crush injuries when upstream regions of undamaged axons were stimulated (panel b of FIG. 7). This was again confirmed by translating the INS probe to fascicles that were damaged distally from the point of stimulation and observing a loss in amplitude and/or increase in latency (panel b of FIG. 12). The distinct clustering seen in the patrial crush condition throughout FIG. 7 and subsequent figures results from separate experiments producing especially consistent amplitudes and latencies. This consistency is likely due to a combination of the positioning of the probe with respect to the injury and maintaining a stable degree of contact between the nerve and stimulation electrode for ES or maintaining a constant distance between the nerve and optical fiber for INS. Overall, crush injuries are more successfully recognized using amplitude rather than latency.

Infrared neural stimulation surpasses electrical stimulation in stretch injury detection

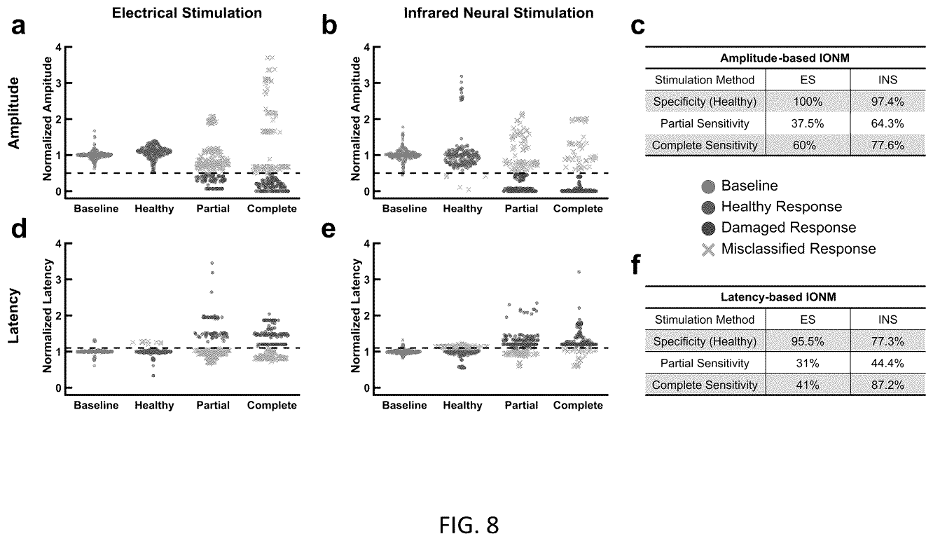

Prior to stretching the nerve, two marks about 2 mm apart were made on the nerve of interest using a surgical ink marker and the distance between the marks was measured using calipers. Stretch injuries were then induced using hook electrodes mounted to micro-manipulators. After putting the nerve in tension with the micro-manipulators, the distance between the marks was remeasured to calculate the strain. Partial and complete stretch injuries had an average strain of 8.6 and 13.4% respectively. For the partial stretch injury, strains of about 8% were chosen as multiple studies found strains between 5 and 10% to be the threshold for functional deficits resulting from stretch injuries. Similarly, for the complete stretch condition, strains above 10% were selected since 10% strains had been previously shown to exceed the nerve's mechanical tolerance. INS yields a higher sensitivity to both partial and complete stretch than ES using both latency- and amplitude-based IONM (panels c and f of FIG. 8). In particular, INS achieves a twofold increase in sensitivity in detecting complete stretch injuries using latency. The difference in sensitivities between the two modalities, however, is not as significant overall as compared with transection and crush injuries. Following stretch injuries, nerves often exhibited greater CMAP amplitudes than observed at baseline (panels a-b of FIG. 8). The specificity of INS appears to suffer using latency-based IONM compared to ES (panels d-e of FIG. 8). Observing the discrepancies in amplitude and latency values in undamaged nerves (i.e., between the baseline and healthy conditions) for both ES and INS, the baseline variance were examined.

Infrared Neural Stimulation Provides More Consistent Amplitude and Latencies in Undamaged Nerves

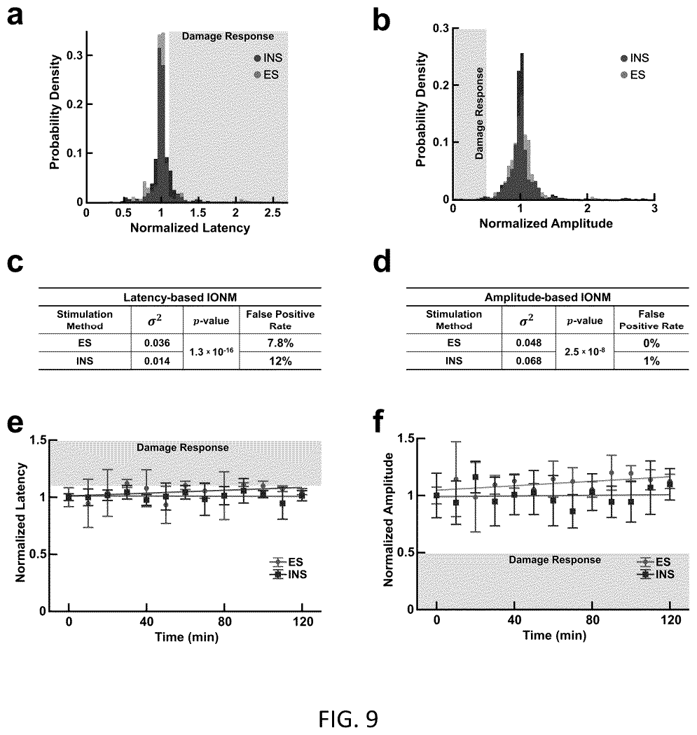

Baseline and healthy data across all experiments were pooled to compare the variability in latency and amplitude of undamaged nerves (panels a-b of FIG. 9). The probability density function of latency and amplitude in undamaged nerves for both ES and INS is not drawn from a normal distribution (p=0 for all cases). Consequently, an Ansari-Bradley test was employed to test for equal variances (i.e., σ²). In undamaged nerves, the test showed that ES and INS latencies possess unequal variances with INS exhibiting a smaller variance (panel c of FIG. 9). ES produces a lower false positive rate (FPR) of 7.8% compared to 12% FPR of INS. Similarly for amplitude, ES and INS again have statistically distinct variances with ES having a significantly smaller standard deviation (panel d of FIG. 9). Despite having unequal variances, both ES and INS share comparable FPR for amplitude. The FPRs for ES and INS were minimal at 0 and 1% respectively. In addition to examining baseline variability, the presence of any time dependent variance was also investigated.

Undamaged nerves were routinely stimulated using ES and INS for 2 hours while changes in latency and amplitude were monitored as seen in panels e and f of FIG. 9. The 2 hour period was chosen to correspond not only with the maximum experiment duration but also exceed the average duration of a conventional thyroidectomy (about 94 min). The magnitude of the latency values for both techniques remained largely constant over the course of the 2 hours (panels e of FIG. 9). When all data points from the latency time course are examined, both INS and ES have nearly identical FPR of 0 and 0.5% respectively. In examining the time course of evoked amplitudes, ES and INS yielded the same FPR of 0.26%.

Given that INS offers comparable if not superior consistency to ES in addition to providing higher sensitivities, the performance of INS in conjunction with a clinical IONM system was then evaluated to ensure comparable efficacy and ease of integration.

### INS is Readily Incorporated Into Existing Clinical IONM Systems Without Loss of Efficacy

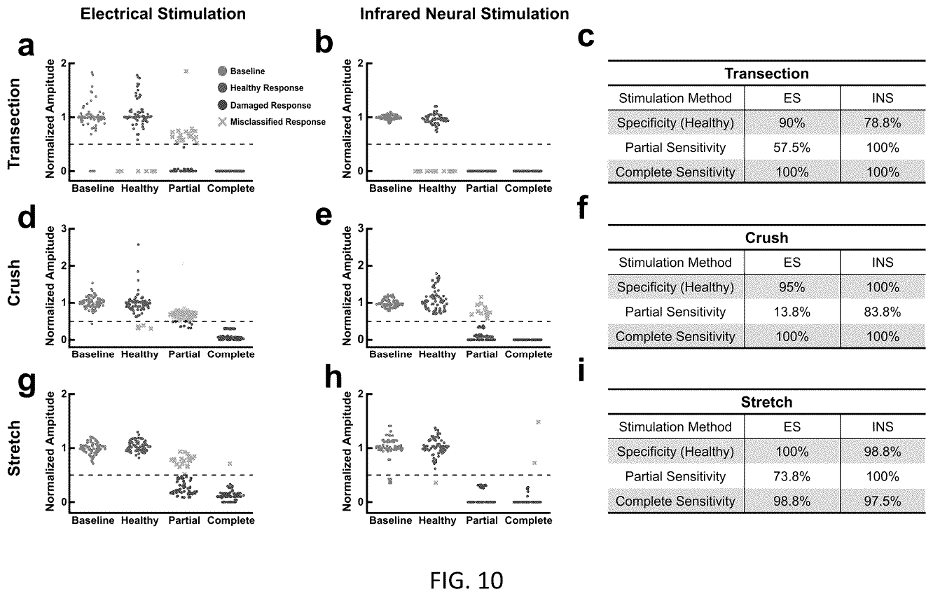

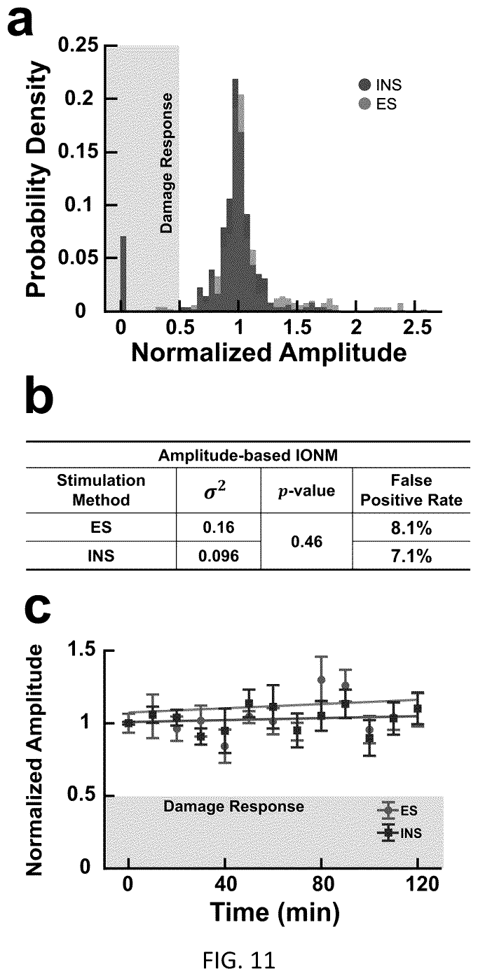

Using a Medtronic NIM-Response 2.0, the same degrees and types of nerve injury were investigated to compare INS and clinical ES without any modifications to the system (FIG. 10). Since INS does not produce a stimulation artifact, latencies could not be accurately measured using the NIM 2.0. Hence, only amplitude-based IONM was examined using the clinical system. Similar to the benchtop system, INS detects all partial transections almost doubling the sensitivity of ES (panels a-c of FIG. 10) similar to what was observed using the benchtop system. As expected, both techniques accurately identified all complete transections. The specificity of ES, however, was substantially higher than that of INS in transection experiments which is more 20 thoroughly explored in FIG. 11. When examining crush injuries, INS vastly outperformed ES in identifying the onset of partial crush with a sensitivity of 83.8 and 13.8% respectively while both correctly classified all full crush CMAPs as damaged (panels d-f of FIG. 10). Trials in which INS failed to detect partial crush injuries were again due to its spatial selectivity (see panel b of FIG. 12). For crush injuries, both INS and ES shared nearly equivalent specificities. For stretch injuries, INS surpasses or matches the sensitivity of ES as seen with the benchtop system. In addition to comparable specificities, INS and ES also produced practically equal sensitivities to complete stretch injuries (panels g-i of FIG. 10). The sensitivity of ES to partial stretch injury did suffer while INS maintained a consistent level of sensitivity for both the partial and full conditions as was the case using the benchtop system.

Baseline and healthy amplitudes across each trial using the clinical IONM system were combined to analyze the variance of both techniques in undamaged nerves. The probability density function of these amplitudes is depicted in panel a of FIG. 11. The amplitudes in undamaged nerves for both ES and INS were not normally distributed (p=0 for all cases). Subsequent testing for variance equivalence revealed the two distributions do not have significantly different variances (panel b of FIG. 11). Though the two techniques derive from similar distributions, INS still produced a smaller variance than ES. In addition to having statistically equivalent variances, the FPRs for INS and ES were 7.1 and 8.1% respectively.

Changes in variance were also examined over time.

Undamaged nerves were routinely stimulated using ES and INS for 2 hours while changes in amplitude were monitored using a clinical IONM system (panel c of FIG. 11). Overall, amplitude values remained relatively consistent over the course of the 2 hour period. Both techniques had an equivalent FPR of 2% across that time frame.

### Discussion

Iatrogenic nerve injury (INI) is a dreaded complication amongst surgeons across disciplines that detrimentally affects patient, provider, and hospital. In procedures where INIs are readily possible or result in severe complications, IONM has become standard practice. During IONM, physiological signals from the nerve's effector are checked for signs of damage after being electrically stimulated. Multiple studies have shown that IONM has reduced the incidence of INIs. Since current IONM relies on ES to generate the necessary evoked potentials, IONM is limited by the inherent limitations of ES namely: current spread, the presence of ES artifacts, and tissue contact which can lead to erroneous results. Due to its high spatial specificity as well as artifact and contact free nature, INS overcomes many of these obstacles and has been touted as both a promising alternative to ES and means to improve IONM. Most studies to date, however, have shown that INS can elicit relevant signals safely, stopping short of providing evidence of INS' clinical value. In this exemplary study, INS was directly compared to ES using both benchtop and clinical IONM systems before and after different types of nerve injury in vivo in an animal model. Using the clinical thresholds for nerve damage detection and examining the three most reported types of INI, the results show that INS surpasses ES in partial injury detection while maintaining similar efficacy and consistency for complete injuries. In the case of partial injuries, INS vastly outperforms ES.

For partial transections, INS exhibited sensitivities over two times higher than ES using both amplitude- and latency-based IONM. This improvement also held when using the clinical IONM system. Though the sensitivity of ES did improve to 58% when using the clinical system, the sensitivity of INS, however, was almost twice as high at 100%. Hence, INS offers tremendous improvement over ES which produced sensitivities as low as 20%. With sensitivities higher than ES, this trend was also apparent for partial crush injuries using both the benchtop and clinical systems. The poor performance of ES in detecting partial crush and transections is likely due to current spread during which unconfined electrical stimulus activates surrounding intact axons still capable of generating adequate CMAPs. Due to its innate spatial selectivity, INS is more sensitive to partial transection and crush injuries. In recruiting a smaller population of axons, damage to fewer axons will result in a more discernible change in the evoked response. This spatial selectivity, however, can also lead to false negatives as seen in panel b of FIG. 6 (and again in panels b and d of FIG. 7) when nondamaged portions of the nerve are stimulated (also see FIG. 12). Hence, looking towards clinical translation, the ability to target individual fascicles or specific portions of nerves will be essential for optimal efficacy and can be easily achieved using multifiber arrays or additional optics. In contexts where the spatial specificity of INS is not desired or larger targets are of interest, the spatial precision of INS can be modified. By adjusting the wavelength and spot size used for stimulation, the stimulated volume can be tailored to specific applications. For partial stretch injuries, the difference in sensitivities between INS and ES was not as stark as with transection and crush.

Using amplitude, there was only 26% difference in sensitivities of INS and ES for partial stretch injuries in both the benchtop and clinical system. INS did attain a sensitivity of 100% using the clinical system, however. Using latency, the difference was only 13% with INS achieving the higher sensitivity of 44%. In general, both techniques poorly detected the presence of partial stretch injuries. This may be attributable to the fact that the strains applied for partial stretch injuries were in most cases recoverable and possibly insufficient to drastically affect amplitude and latency of the evoked CMAPs which has been observed in previous studies. The smaller difference in sensitivity between INS and ES may also be a consequence of the entire nerve being stretched rather than a fraction of its diameter. Hence, the spatial precision of INS does not contribute to its diagnostic accuracy. Both modalities are probing stretched axons which appears to be a difficult type of damage to classify using latency and/or amplitude. Moreover, with stretch injuries, some amplitudes evoked after stretching were substantially higher than those at baseline (panels a-b of FIG. 8). Other studies have reported similar findings and provide evidence that stretch injuries increase excitability as well as generate greater amplitude CMAPs especially during recovery periods as short as 3 minutes. Thus, it is possible some nerves had sufficient time to recover between stretching and stimulation. It should also be noted that the stretch injuries inflicted here cannot be completely decoupled from the trauma caused by the hooks used to stretch the nerve. The IONM results for both stimulation techniques after stretch, however, are quite distinct from both transection and crush injuries as stated previously. Since the hooks would likely cause a compression or crush injury, this suggests that a different type of damage (i.e., stretch) is occurring. Despite both techniques leaving room for improvement in the detection of partial stretch injuries, INS dependably identified more injuries than ES using both nerve monitoring systems and metrics (i.e., amplitude and latency). In moving from partial to complete forms of damage, however, the efficacy of INS and ES were largely on par. As expected, ES and INS correctly identified all complete transections using both the benchtop and clinical systems. For complete crush injuries, the performance of the two techniques was also comparable. With the benchtop system, however, INS correctly classified every evoked response as damaged while ES reached a sensitivity of about 80% using amplitude and latency-based IONM. Both techniques attained sensitivities of 100% using the clinical IONM system. Compared to the partial condition, the sensitivity for both ES and INS increase slightly for complete stretch injuries using the benchtop system. Nonetheless, INS again produced higher sensitivities than ES except when using the clinical system which yielded nearly equivalent sensitivities. For each complete injury type, INS often exceeded or at the very least matched the efficacy of ES using both IONM systems and approaches. Additionally, INS consistently achieved a higher sensitivity than ES for both degrees of severity, IONM systems, and approaches. While the efficacy of INS surpassed that of ES, amplitude-based IONM also generally provided more accurate classification than latency-based IONM.

Taken as a whole, latency-based IONM regularly underperformed compared to the amplitude-based approach. Latency-based IONM only bettered amplitude-based once in recognizing complete stretch injuries using INS. Moreover, for complete transections and crush injuries, latency-based sensitivities matched that of amplitude-based exclusively when no CMAPs were evoked. Although latency was only measured with the benchtop system, these results seem to suggest that amplitude-based IONM offers a more robust and accurate indication of nerve health and functionality. This may also account for reason many surgeons only utilize amplitude-based IONM rather than latency alone or a combination of the two. In addition to investigating the sensitivity of INS during IONM, the specificity and consistency of INS-induced amplitudes and latencies was also analyzed over time and across systems.

The statistical analysis of ES- and INS-induced latency distributions in undamaged nerves revealed that both have unequal variances (panel c of FIG. 9). Of the two, INS had the smaller variance and consequently a standard deviation 7% lower than that of ES. Since the latency damage threshold is defined as a 10% increase from baseline, this suggests that having a smaller standard deviation even by 7% could improve nerve damage detection and reduce the risk of false positives. Extended monitoring of undamaged nerves over a period of two hours showed that INS and ES induced latencies had equal FPR over the duration (panel c of FIG. 9). INS did, however, exhibit a higher FPR than ES in the short term (<10 min after baseline acquisition) based on the data from nerve injury trials. Overall, this suggests that INS produces comparable if not more consistent latencies over time than ES. This trend was also observed in INS- and ES-induced amplitudes.

Both ES and INS amplitude distributions also have statistically different variances for the bench top system with ES has having a smaller overall standard deviation by 4% (panel d of FIG. 9). In relation to the 50% decrease in amplitude damage threshold, this difference is variance is insignificant. Accordingly, both techniques have statistically equivalent variances when using the clinical IONM system. Using both systems, each technique had FPRs differing less than a percent across all considered time frames (panel d of FIG. 9 and panel b of FIG. 11). Variability in ES amplitude data is likely due to slight variations in electrode's contact with the nerve (panel a of FIG. 6) while the lower variability of INS mediated CMAPs is possibly due to the fact INS is non-contact. Hence, the efficacy of INS may be less susceptible to probe placement and manipulations of the surgical field as long as its spatial selectivity is well managed. Since water absorption of infrared light is the driving mechanism underlying INS, the primary source of variability in INS data is likely due to changes in tissue hydration. Taken together, INS provides more consistent amplitudes and latencies in undamaged nerves and provides comparable if not less variability than ES.

By integrating INS into a clinical IONM system, we also took the first step to show that the benefits INS offers are readily translatable to existing IONM systems. The results confirm that INS is easily incorporated into clinical IONM systems already in use during surgery without a loss in efficacy. This provides a clear path for INS into the operating room with minimal disruption to current surgical workflows. If quantification of latency is desired, additional modifications to current clinical IONM would need to be made to allow for accurate recordings of both the optical stimulus and evoked signal. Given that latency-based IONM seems to frequently provide erroneous classifications and some surgeons chose to rely solely on amplitude, these modifications may not be in high demand.

Using the clinical thresholds for nerve damage detection, we have demonstrated that INS, a safe and proven neurostimulation method, is more sensitive to partial forms of damage than clinical ES and exhibits equal if not superior sensitivity to more severe injuries. The enhanced sensitivity of INS is largely due to its high degree of inherent spatial selectivity. With improved sensitivity to nerve injury, surgeons could be alerted to the onset of damage earlier preventing further trauma and enabling timely interventions. Moreover, INS largely yields more consistent and reliable latencies and amplitudes in undamaged nerves. Hence, in surgery, INS has the potential to provide more consistent, reliable values and clearer, more accurate indications of nerve damage. Able to be readily integrated into current clinical IONM systems, the findings of this study substantiate the clinical value of INS for IONM and propose a simple means to improve surgical outcomes by sparing both patients and surgeons from the adverse effects of INIs.

The superior accuracy of INS for detecting partial injuries is likely due to its inherent spatial precision. By monitoring a subset of the axons comprising the nerve, INS is more sensitive to the onset of less severe forms of damage that may otherwise be obscured when stimulating the whole nerve. For future clinical applications, multiplexed INS systems can be designed to monitor all axon populations simultaneously.

The foregoing description of the exemplary embodiments of the present invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

1. Antoniadis, G. et al. Iatrogenic nerve injuries: Prevalence, diagnosis and treatment. Dtsch. Arztebl. Int. 111, 273-279. https://doi.org/10.3238/arzteb1.2014.0273 (2014).

2. Burke, S. & Shorten, G. D. When pain after surgery doesn't go away . . . . Biochem. Soc. Trans. 37, 318-322. https://doi.org/10.1042/BST0370318 (2009).

3. Ficarra, V., Novara, G., Rosen, R. C., Artibani, W., Carroll, P. R., Costello, A. et al. Systematic review and meta-analysis of studies reporting urinary continence recovery after robot-assisted radical prostatectomy. https://doi.org/10.1016/j.eururo.2012.05.045 (n.d).

4. Emanu, J. C., Avildsen, I. K., Nelson, C. J. Erectile dysfunction after radical prostatectomy: prevalence, medical treatments, and psychosocial interventions. https://doi.org/10.1097/SPC.0000000000000195 (n.d).

5. Randolph, G. W. & Kamani, D. Intraoperative electrophysiologic monitoring of the recurrent laryngeal nerve during thyroid and parathyroid surgery: Experience with 1,381 nerves at risk. Laryngoscope 127, 280-286. https://doi.org/10.1002/lary.26166 (2017).

6. Kehlet, H., Jensen, T. S. & Woolf, C. J. Persistent postsurgical pain: Risk factors and prevention. Lancet 367, 1618-1625. https://doi.org/10.1016/S0140-6736(06)68700-X (2006).

7. Hall, M. J., DeFrances, C. J., Williams, S. N., Golosinskiy, A. & Schwartzman, A. National Hospital Discharge Survey: 2007 summary. Natl. Health Stat. Rep. 1-20, 24 (2010).

8. Defrances, C. J., Lucas, C. A., Buie, V. C., Golosinskiy, A. National Health Statistics Reports Number 5 (2006).

9. Defrances, C. J., Hall, M. J. Vital and health statistics. Adv. Data 385 (2005)

10. Schover, L. R. et al. Defining sexual outcomes after treatment for localized prostate carcinoma. Cancer https://doi.org/10.1002/cncr.10848 (2002).

11. Gartner, R., Jensen, M.-B., Nielsen, J., Ewertz, M., Kroman, N., Kehlet, H. Prevalence of and factors associated with persistent pain following breast cancer surgery (n.d).

12. Delank, K. S. et al. Iatrogenic paraplegia in spinal surgery. Arch. Orthop. Trauma Surg. 125, 33-41. https://doi.org/10.1007/s00402-004-0763-5 (2005).

13. Kaylie, D. M., Gilbert, E., Horgan, M. A., Delashaw, J. B. & McMenomey, S. 0. Acoustic Neuroma Surgery Outcomes. Otol Neurotol 22, 686-689 (2001).

14. Abadin, S. S., Kaplan, E. L. & Angelos, P. Malpractice litigation after thyroid surgery: The role of recurrent laryngeal nerve injuries, 1989-2009. Surgery 148, 718-723. https://doi.org/10.1016/J.SURG.2010.07.019 (2010).

15. Barczyliski, M. et al. Intraoperative nerve monitoring can reduce prevalence of recurrent laryngeal nerve injury in thyroid reoperations: Results of a retrospective cohort study. World J. Surg. 38, 599-606. https://doi.org/10.1007/s00268-013-2260-x (2014).

16. Campbell, W. W. Evaluation and management of peripheral nerve injury. Clin. Neurophysiol. 119, 1951-1965. https://doi.org/10.1016/J.CLINPH.2008.03.018 (2008).

17. Kern, K. A. Medicolegal analysis of errors in diagnosis and treatment of surgical endocrine disease. Surgery 114, 1167-1173.

https://doi.org/10.5555/URIPII:003960609390340J (1993).

18. Morris, L. G. T., Ziff, D. J. S. & DeLacure, M. D. Malpractice litigation after surgical injury of the spinal accessory nerve. Arch. Otolaryngol. Neck Surg. 134, 102. https://doi.org/10.100 1 / a rchoto1.134.1.102 (2008).

19. Winfree, C. J. & Kline, D. G. Intraoperative positioning nerve injuries. Surg. Neurol. 63, https://doi.org/10.1016/J.SURNEU.2004.03.024 (2005).

20. Brown, R. H. & Nash, C. L. Current status of spinal cord monitoring. Spine (Phila Pa 1976) 4, 466-470 (1979).

21. Nash, C. L., Lorig, R. A., Schatzinger, L. A. & Brown, R. H. Spinal cord monitoring during operative treatment of the spine. Clin. Orthop. Relat. Res. 126, 100-105 (1977).

22. Barczyński, M., Konturek, A. & Cichon, S. Randomized clinical trial of visualization versus neuromonitoring of recurrent laryngeal nerves during thyroidectomy. Br. J. Surg. 96, 240-246. https://doi.org/10.1002/bjs.6417 (2009).

23. Wong, K. P., Mak, K. L., Wong, C. K. H. & Lang, B. H. H. Systematic review and meta-analysis on intra-operative neuro-monitoring in high-risk thyroidectomy. Int. J. Surg. 38, 21-30. https://doi.org/10.1016/J.IJSU.2016.12.039 (2017).

24. Dionigi, G. et al. Continuous monitoring of the recurrent laryngeal nerve in thyroid surgery: A critical appraisal. Int. J. Surg. 11, S44—S46. https://doi.org/10.1016/S1743-9191(13)60014-X (2013).

25. Sarnthein, J., Tomilov, M., Baag, M. & Regli, L. Improving intraoperative evoked potentials at short latency by a novel neuro-stimulation technology with delayed return discharge. Clin. Neurophysiol. 132, 1195-1199 (2021).

26. Testerman, R. L. Comments on ‘Accuracy limitations of chronaxie values’. IEEE Trans. Biomed. Eng. https://doi.org/10.1109/TBME.2004.836506 (2005).

27. Fried, N. M. et al. Identification and imaging of the nerves responsible for erectile function in rat prostate, in vivo, using optical nerve stimulation and optical coherence tomography. IEEE J. Sel. Top. Quantum Electron. 13, 1641-1645. https://doi.org/10.1109/JSTQE.2007.910119 (2007).

28. Kim, H. L., Stoffel, D. S., Mhoon, D. A. & Brendler, C. B. A positive caver map response poorly predicts recovery of potency after radical prostatectomy. Urology 56, 561-564. https://doi.org/10.1016/S0090-4295(00)00748-2 (2000).

29. Wells, J., Konrad, P., Kao, C., Jansen, E. D. & Mahadevan-Jansen, A. Pulsed laser versus electrical energy for peripheral nerve stimulation. J. Neurosci. Methods 163, 326-337. https://doi.org/10.1016/J.JNEUMETH.2007.03.016 (2007).

30. Moser, T. & Dieter, A. Towards optogenetic approaches for hearing restoration. Biochem. Biophys. Res. Commun. 527, 337-342. https://doi.org/10.1016/J.BBRC.2019.12.126 (2020).

31. Song, W. H. et al. Establishment of novel intraoperative monitoring and mapping method for the cavernous nerve during robot-assisted radical prostatectomy: Results of the phase VII, first-in-human, feasibility study. Eur. Urol. https://doi.org/10.1016/J.EURUR0.2019.04.042 (2019).

32. Fried, N. M. & Burnett, A. L. Novel methods for mapping the cavernous nerves during radical prostatectomy. Nat. Rev. Urol. 12, 451-460. https://doi.org/10.1038/nruro1.2015.174 (2015).

33. Wells, J., Kao, C., Jansen, E. D., Konrad, P. & Mahadevan-Jansen, A. Application of infrared light for in vivo neural stimulation. J. Biomed. Opt. 10, 064003. https://doi.org/10.1117/1.2121772 (2005).

34. Cayce, J. M. et al. Infrared neural stimulation of primary visual cortex in non-human primates. Neuroimage 84, 181-190. https://doi.org/10.1016/INEUROIMAGE.2013.08.040 (2014).

35. Cayce, J. M. et al. Infrared neural stimulation of thalamocortical brain slices. IEEE J. Sel. Top. Quantum Electron. 16, 565-572. https://doi.org/10.1109/JSTQE.2009.2032424 (2010).

36. Zhao, K., Tan, X., Young, H., Richter, C.-P. Stimulation of neurons with infrared radiation. Biomed. Opt. Otorhinolaryngol. 253-84 (2016).

37. Throckmorton, G. et al. Identifying optimal parameters for infrared neural stimulation in the peripheral nervous system. Neurophotonics 8, 015012. https://doi.org/10.1117/1.nph.8.1.015012 (2021).

38. Wells, J. et al. Biophysical mechanisms of transient optical stimulation of peripheral nerve. Biophys. J. https://doi.org/10.1529/biophysj.107.104786 (2007).

39. Shapiro, M. G., Homma, K., Villarreal, S., Richter, C.-P. & Bezanilla, F. Infrared light excites cells by changing their electrical capacitance. Nat. Commun. https://doi.org/10.1038/ncomms1742 (2012).

40. Plaksin, M., Shapira, E., Kimmel, E. & Shoham, S. Thermal transients excite neurons through universal intramembrane mechanoelectrical effects. Phys. Rev. X8, 011043. https://doi.org/10.1103/PhysRevX.8.011043 (2018).

41. Cayce, J. M. et al. Infrared neural stimulation of human spinal nerve roots in vivo. Neurophotonics 2, 015007. https://doi.org/10.1117/1.NPh.2.1.015007 (2015).

42. Matic, A. I., Walsh, J. T. & Richter, C.-P. Spatial extent of cochlear infrared neural stimulation determined by tone-on-light masking. J. Biomed. Opt. 16, 118002. https://doi.org/10.1117/1.3655590 (2011).

43. Jenkins, M. W. et al. Optical pacing of the embryonic heart. Nat. Photonics 4, 623-626. https://doi.org/10.1038/nphoton.2010.166 (2010).

44. Jenkins, M. W. et al. Optical pacing of the adult rabbit heart. Biomed. Opt. Express 4, 1626-1635. https://doi.org/10.1364/BOE.4.001626 (2013).

45. Prodanov, D., Feirabend, H. K. P. & Feirabend, H. K. P. Morphometric analysis of the fiber populations of the rat sciatic nerve, its spinal roots, and its major branches. J. Comp. Neurol. 503, 85-100. https://doi.org/10.1002/cne.21375 (2007).

46. Serpell, J. W., Woodruff, S., Bailey, M., Grodski, S. & Yeung, M. Recurrent laryngeal nerve diameter increases during thyroidectomy. Ann. Surg. Oncol. 18, 1742-1747. https://doi.org/10.1245/s10434-010-1531-0 (2011).

47. Lo, Y. L. et al. High-resolution ultrasound in the evaluation and prognosis of Bell's palsy. Eur. J. Neurol. 17, 885-889. https://doi.org/10.1111/j.1468-1331.2010.02950.x (2010).

48. Kretschmer, T., Heinen, C. W., Antoniadis, G., Richter, H.-P. & Konig, R. W. Iatrogenic nerve injuries. Neurosurg. Clin. N. Am. 20, 73-90. https://doi.org/10.1016/J.NEC.2008.07.025 (2009).

49. Hussain, G. et al. Current status of therapeutic approaches against peripheral nerve injuries: A detailed story from injury to recovery. Int. J. Biol. Sci. 16, 116-134. https://doi.org/10.7150/IJBS.35653 (2020).

50. Schneider, R. et al. Continuous intraoperative neural monitoring of the recurrent nerves in thyroid surgery: A quantum leap in technology. Gland Surg. 5, 607-616. https://doi.org/10.21037/gs.2016.11.10 (2016).

51. Toleikis, J. R. & Abnm, D. Intraoperative monitoring using somatosensory evoked potentials a position statement by the American Society of Neurophysiological Monitoring. J. Clin. Monit. Comput. 19, 241-258. https://doi.org/10.1007/s10877-005-4397-0 (2005).

52. Rickett, T., Connell, S., Bastijanic, J., Hegde, S. & Shi, R. Functional and mechanical evaluation of nerve stretch injury. J. Med. Syst. 35, 787-793. https://doi.org/10.1007/S10916-010-9468-1 (2011).

53. Driscoll, P. J., Glasby, M. A. & Lawson, G. M. An in vivo study of peripheral nerves in continuity: Biomechanical and physiological responses to elongation. J. Orthop. Res. 20, 370-375. https://doi.org/10.1016/S0736-0266(01)00104-8 (2002).

54. Li, J. & Shi, R. A device for the electrophysiological recording of peripheral nerves in response to stretch. J. Neurosci. Methods 154, 102-108. https://doi.org/10.1016/J.JNEUMETH.2005.12.007 (2006).

Yao, H. S., Wang, Q., Wang, W. J. & Ruan, C. P. Prospective clinical trials of thyroidectomy with LigaSure vs conventional vessel ligation: A systematic review and meta-analysis. Arch. Surg. 144, 1167-1174. https://doi.org/10.1001/archsurg.2009.201 (2009).

56. Kwan, M. K., Wall, E. J., Massie, J. & Garfin, S. R. Strain, stress and stretch of peripheral nerve Rabbit experiments in vitro and in vivo. Acta Orthop. Scand. 63, 267- 272. https://doi.org/10.3109/17453679209154780 (1992).

57. Stecker, M. M., Baylor, K., Wolfe, J. & Stevenson, M. Acute nerve stretch and the compound motor action potential. J. Brachial Plexus Peripher. Nerve Injury 66, ell—e22 (2011).

58. Wells, J. D. et al. Optically mediated nerve stimulation: Identification of injury thresholds. Lasers Surg. Med. 39, 100. https://doi.org/10.1002/1sm.20522 (2007).

59. Khosrofian, J. M. & Garetz, B. A. Measurement of a Gaussian laser beam diameter through the direct inversion of knife-edge data. Appl. Opt. 22, 3406. https://doi.org/10.1364/A0.22.003406 (1983).

60. Fried N M, Lagoda G A, Scott N J, Su L-M, Burnett A L. Laser Stimulation of the Cavernous Nerves in the Rat Prostate, in Vivo: Optimization of Wavelength, Pulse Energy, and Pulse Repetition Rate.

61. Kaouk G S, Perkins W C, Lagoda G A, Burnett A L, Fried N M. Optical and Electrical Stimulation of the Rat Prostate Cavernous Nerves: Priming and Fatigue Studies.

62. Tozburun S, Lagoda G A, Burnett A L, Fried N M. Infrared Laser Nerve Stimulation as a Potential Diagnostic Method for Intra-Operative Identification and Preservation of the Prostate Cavernous Nerves. IEEE J Sel Top Quantum Electron 2014;20:299-306. https://doi.org/10.1109/JSTQE.2013.2293273.

63. Tozburun S, Lagoda G A, Burnett A L, Fried N M. Continuous-Wave Laser Stimulation of the Rat Prostate Cavernous Nerves Using a Compact and Inexpensive All Single Mode Optical Fiber System. J Endourol 2011;25:1727-31. https://doi.org/10.1089/end.2011.0172.

64. Tozburun S, Mayeh M, Lagoda G A, Farahi F A, Burnett A L, Fried N M. A Compact Laparoscopic Probe for Optical Stimulation of the Prostate Nerves. IEEE J Sel Top Quantum Electron 2010;16:941-5. https://doi.org/10.1109/JSTQE.2009.2035432.

65. Tozburun S, Hutchens T C, McClain M A, Lagoda G A, Burnett A L, Fried N M. Temperature-controlled optical stimulation of the rat prostate cavernous nerves. J Biomed Opt 2013;18:067001. https://doi.org/10.1117/1.JBO.18.6.067001.

66. Horiguchi A, Tsujita K, Irisawa K, Kasamatsu T, Hirota K, Kawaguchi M, et al. A pilot study of photoacoustic imaging system for improved real-time visualization of neurovascular bundle during radical prostatectomy. Prostate 2016;76:307-15. https://doi.org/10.1002/pros.23122.

67. Fried N M, Rais-Bahrami S, Lagoda G A, Chuang A-Y, Su L-M, Burnett III A L. Identification and Imaging of the Nerves Responsible for Erectile Function in Rat Prostate, In Vivo, Using Optical Nerve Stimulation and Optical Coherence Tomography. IEEE J Sel Top Quantum Electron 2007;13:1641-5. https://doi.org/10.1109/JSTQE.2007.910119.

68. Tozburun S, Lagoda G A, Burnett A L, Fried N M. Gaussian versus Flat-Top Spatial Beam Profiles for Optical Stimulation of the Prostate Nerves.

69. Fried N M, Lagoda G A, Scott N J, Su L-M, Burnett A L. Noncontact stimulation of the cavernous nerves in the rat prostate using a tunable-wavelength thulium fiber laser. J Endourol 2008;22:409-14.

70. Perkins W C, Lagoda G A, Burnett A, Fried N M. Electrical Stimulation vs. Pulsed and Continuous-Wave Optical Stimulation of the Rat Prostate Cavernous Nerves, in Vivo.

71. Tozburun S, Lagoda G A, Burnett A L, Fried N M. Subsurface near-infrared laser stimulation of the periprostatic cavernous nerves. J Biophotonics 2012;5:793-800. https://doi.org/10.1002/jbio.201100134.

72. Teudt I U, Nevel A E, Izzo A D, Walsh J T, Richter C-P. Optical Stimulation of the Facial Nerve: A New Monitoring Technique? Laryngoscope 2007;117:1641-7. https://doi.org/10.1097/MLG.0b013e318074ec00.

73. Wells J, Konrad P, Kao C, Jansen E D, Mahadevan-Jansen A. Pulsed laser versus electrical energy for peripheral nerve stimulation. J Neurosci Methods 2007;163:326-37.

https://doi.org/10.1016/J.JNEUMETH.2007.03.016.

74. Cayce J M, Wells J D, Malphrus J D, Kao C, Thomsen S, Tulipan N B, et al. Infrared neural stimulation of human spinal nerve roots in vivo. Neurophotonics 2015;2:015007. https://doi.org/10.1117/1.NPh.2.1.015007.

75. Throckmorton G, Cayce J, Ricks Z, Adams W R, Jansen E D, Mahadevan-Jansen A. Identifying optimal parameters for infrared neural stimulation in the peripheral nervous system. Neurophotonics 2021;8:015012. https://doi.org/10.1117/1.nph.8.1.015012.

What is claimed is:

1. A system of neural stimulation for intraoperative nerve monitoring for a living subject, comprising:
an optical source configured to generate light;
a delivering means coupled to the optical source to deliver the generated light to a target nerve of the living subject for stimulating the target nerve; and
a detector coupled to the target nerve to record evoked signals responsive to the stimulation for intraoperatively monitoring of the target nerve.

2. The system of claim 1, wherein the light source comprises a laser.

3. The system of claim 2, wherein the laser comprises a pulsed infrared laser.

4. The system of claim 3, wherein the light is pulsed infrared light having a wavelength in a range of about 1000-2500 nm, and a pulse duration in a range of about 1-10 ms.

5. The system of claim 4, wherein the pulsed infrared light has a pulse energy in a range of about 1-25 mJ with a radiant exposure in a range of about 0.1-3 J/cm².

6. The system of claim 1, wherein the delivering means is adapted for delivering the light directly to the target nerve at a distance away from the surface of the target nerve.

7. The system of claim 6, wherein the delivering means comprises a probe having one end coupled to the optical source for receiving the light therefrom and an opposite, working end for delivering the light to the target nerve, and wherein the working end is positioned at the distance away from the surface of the target nerve such that there is no object positioned between the working end of the probe and the target nerve.

8. The system of claim 7, wherein the distance is in a range of about 10-500 μm.

9. The system of claim 7, wherein the probe comprises one or more optical fibers, one or more wave guides, one or more channels, or a combination thereof.

10. The system of claim 7, wherein the delivering means further comprises a movable stage coupled to the probe for adjustably positioning the working end of the probe at the distance away from the target nerve.

11. The system of claim 10, wherein the movable stage comprises a micromanipulator.

12. The system of claim 6, wherein the delivering means comprises one or more optical mirrors, one or more optical lenses, one or more optical couplers, or a combination thereof, placed in an optical path for focusing and/or collimating the light onto the target nerve.

13. The system of claim 1, wherein the detector comprises at least one sensing electrode placed in a downstream muscle associated with the target nerve for recording the evoked signals.

14. The system of claim 13, wherein the detector is configured to record the evoked signals at a sampling rate in a range of about 5000-8000 Hz.

15. The system of claim 14, wherein the detector is further configured to process the evoked signals to obtain amplitudes and latencies of the evoked signals, wherein each amplitude is a difference between the maximum and minimum of each evoked signal, and wherein each latency is a duration from the peak of the stimulus to the peak of each evoked signal.

16. The system of claim 15, wherein the amplitudes and latencies are normalized to the mean of the corresponding baseline values.

17. The system of claim 16, wherein a ≥50% loss in a baseline amplitude and a ≥10% increase in a baseline latency serve as thresholds for neural damage detection.

18. The system of claim 1, wherein the evoked signals comprise compound muscle action potentials (CMAPs).

19. A method of neural stimulation for intraoperative nerve monitoring for a living subject, comprising:
delivering light to a target nerve of the living subject at a distance away from the target nerve for stimulating the target nerve;
recording evoked signals of the target nerve responsive to the stimulation; and
processing the evoked signals for intraoperatively monitoring of the target nerve.

20. The method of claim 19, wherein the light is generated by an optical source including a pulsed infrared laser.

21. The method of claim 20, wherein the light is pulsed infrared light having a wavelength in a range of about 1000-2500 nm, and a pulse duration in a range of about 1-10 ms.

22. The method of claim 21, wherein the pulsed infrared light has a pulse energy in a range of about 1-25 mJ with a radiant exposure in a range of about 0.1-3 J/cm².

23. The method of claim 20, wherein said delivering the light is performed by a probe having one end coupled to the optical source for receiving the light therefrom and an opposite, working end for delivering the light to the target nerve, and wherein the working end is positioned at a distance away from the surface of the target nerve such that there is no object positioned between the working end of the probe and the target nerve.

24. The method of claim 23, wherein the distance is in a range of about 10-500

25. The method of claim 23, wherein the probe comprises one or more optical fibers, one or more wave guides, one or more channels, or a combination thereof.

26. The method of claim 23, wherein the working end of the probe is adjustably positioned at the distance away from the target nerve by a moveable stage.

27. The method of claim 26, wherein the movable stage comprises a micromanipulator.

28. The method of claim 19, wherein said delivering the light is performed by one or more optical mirrors, one or more optical lenses, one or more optical couplers, or a combination thereof, placed in an optical path for focusing and/or collimating the light onto the target nerve.

29. The method of claim 19, wherein said recording the evoked signals of the target nerve is performed by a detector having at least one sensing electrode placed in a downstream muscle associated with the target nerve for recording the evoked signals.

30. The method of claim 29, wherein the evoked signals of the target nerve is recorded at a sampling rate in a range of about 5000-8000 Hz.

31. The method of claim 30, wherein said processing the evoked signals comprises
obtaining amplitudes and latencies of the evoked signals, wherein each amplitude is a difference between the maximum and minimum of each evoked signal, and wherein each latency is a duration from the peak of the stimulus to the peak of each evoked signal; and
normalizing the amplitudes and latencies to the mean of the corresponding baseline values.

32. The method of claim 31, wherein a ≥50% loss in a baseline amplitude and a ≥10% increase in a baseline latency serve as thresholds for neural damage detection.

33. The system of claim 19, wherein the evoked signals comprise compound muscle action potentials (CMAPs).
